(19) United States
(12) Patent Application Publication
(10) Pub. No.: US 2021/0274952 A1
(43) Pub. Date: Sep. 9, 2021

(54) FIVE POINT HARNESS ACCOMMODATING WEARABLE BLANKET

(71) Applicant: Eva Coe, Rosharon, TX (US)

(72) Inventor: Eva Coe, Rosharon, TX (US)

(21) Appl. No.: 16/813,411

(22) Filed: Mar. 9, 2020

(51) Int. Cl. A47G 9/06; A61F 5/37

(52) U.S. Cl. CPC A47G 9/066; A61F 5/3776

(57) ABSTRACT

A wearable blanket covers a wearer in a five point harness over both shoulders, around a waist and a crotch of the wearer includes a left and a right sleeve and a neck in a shoulder portion and a back portion extending to armpits of the wearer. A torso portion extends from the shoulder portion over a chest and down to the waist of the wearer. A left side flap and a right side flap extend from the torso portion and are available to tuck under a back and a bottom of the wearer. A leg portion extends from the waist of the torso portion and includes a portion of the left and the right side flaps. A feet pocket extends over both legs from the leg portion and configured to enclose the feet and the legs of the wearer. An elastic sustains the feet pocket adjacent the wearer's knees.

### BACKGROUND

A five-point harness is a form of seat belt that contains five straps that are mounted to a chair or other frame. It has been engineered for an increase of safety in the occurrence of accidents and disabilities. As a result, this form of seat belt has been mandated in certain health care industries. This invention has also been used to secure infants and young children in child safety seats.

A five point harness means two belt straps around a person's shoulders, one between their legs and two belts that hold the hips. A three point harness only has the shoulders straps and one strap between a person's legs and therefore lacks the straps on the hips.

The more straps the better harnessing for a passenger or a disabled person. A five point harness secures a person better in their seat, absorbs extreme collisions more and distributes the force on the person's strong parts like their shoulders and hips. In contrast, the majority of tension in a three point harness goes only to the shoulders. Moreover, with no hip straps the child might be ejected out of the seat in a three point harness.

However, all harnesses share the inconvenience to a wearer for getting in and out. Especially cumbersome for winter or cold environments, harnesses restrict bulky clothing and warm blankets. For these reasons and more, there exists a need in the market for a way of insulating persons disabled or using harnesses without getting in and out of the harness for comfort and warmth.

### SUMMARY OF THE INVENTION

A wearable blanket covers a wearer in a five point harness over both shoulders, around a waist and a crotch of the wearer includes a left and a right sleeve and a neck in a shoulder portion and a back portion extending to armpits of the wearer. A torso portion extends from the shoulder portion over a chest and down to the waist of the wearer. A left side flap and a right side flap extend from the torso portion and are available to tuck under a back and a bottom of the wearer. A leg portion extends from the waist of the torso portion and includes a portion of the left and the right side flaps. A feet pocket extends over both legs from the leg portion and configured to enclose the feet and the legs of the wearer. An elastic sustains the feet pocket adjacent the wearer's knees.

The disclosed method for wearing a blanket in a five point harness includes forming a left and a right sleeve and a neck defined in a shoulder portion and a back portion extending to armpits of a wearer. The method also includes attaching a torso portion extending from the shoulder portion over a chest and a lap of a wearer down to the waist of the wearer. The method additionally includes extending a left side flap and a right side flap from the torso portion and available to tuck under a back and a bottom of the wearer. The method further including a leg portion extending from the waist of the torso portion and having a portion of the left and the right side flaps. The method yet includes sustaining a feet pocket extending over both legs from the leg portion and configured to enclose the feet and the legs of the wearer.

### BRIEF DESCRIPTION OF THE DRAWINGS

FIG. 1 depicts a front perspective view of the five point harness accommodating wearable blanket in accordance with an embodiment of the present disclosure.

FIG. 2 depicts a back perspective view of the five point harness accommodating wearable blanket in accordance with an embodiment of the present invention.

FIG. 3 is a side perspective view of the five point harness accommodating wearable blanket in accordance with an embodiment of the present disclosure.

FIG. 4 is a flow chart of a method of wearing the five point harness accommodating wearable blanket in accordance with an embodiment of the present disclosure.

Throughout the description, similar reference numbers may be used to identify similar elements depicted in multiple embodiments. Although specific embodiments of the invention have been described and illustrated, the invention is not to be limited to the specific forms or arrangements of parts so described and illustrated. The scope of the invention is to be defined by the claims appended hereto and their equivalents.

### DETAILED DESCRIPTION

Reference will now be made to exemplary embodiments illustrated in the drawings and specific language will be used herein to describe the same. It will nevertheless be understood that no limitation of the scope of the disclosure is thereby intended. Alterations and further modifications of the inventive features illustrated herein and additional applications of the principles of the inventions as illustrated herein, which would occur to one skilled in the relevant art and having possession of this disclosure, are to be considered within the scope of the invention.

Throughout the present disclosure the term ‘pocket’ is used synonymously with ‘pouch’ and ‘patch’ in the common sense. In the present disclosure, a foot pocket is also a foot pouch for receiving and enclosing the feet of a wearer. Also, a breast pocket is disclosed over a breast area of the wearable blanket. The breast pocket or breast patch is generally a smaller size pocket than the foot pocket or foot pouch.

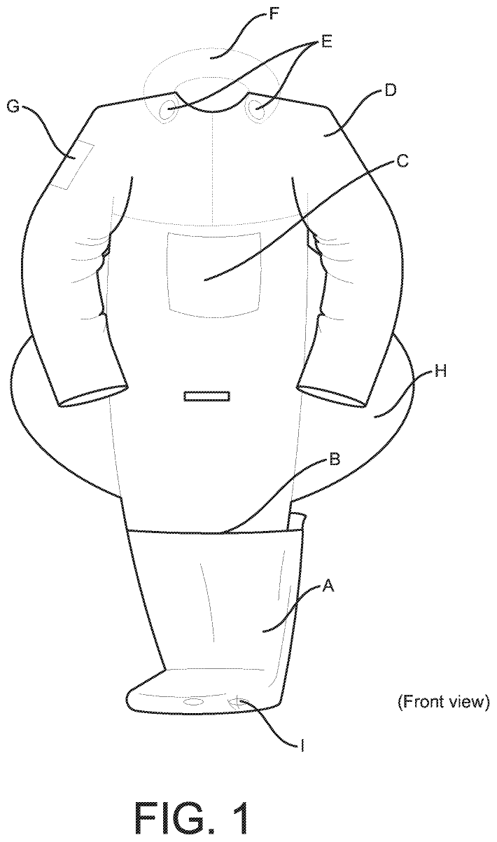

FIG. 1 depicts a front perspective view of the five point harness accommodating wearable blanket in accordance with an embodiment of the present disclosure. The depiction includes a feet pocket A folder over the feet of a wearer and an elastic in the back of the knees. The depiction also includes a big pouch or pocket C, a warm or cool material D, a hook and loop attachment E for a neck roll, a neck roll F in broken lines, a glasses holder or pocket G, a left and a right side flap H and internal pockets I to insert pads for heels of the feet.

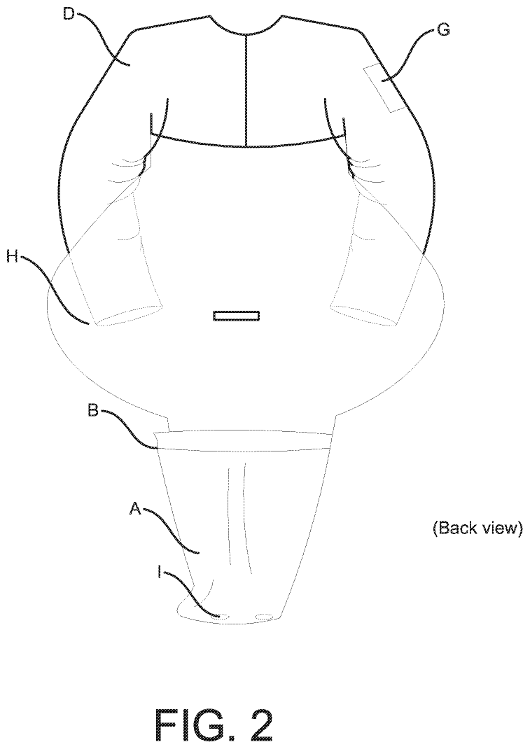

FIG. 2 depicts a back perspective view of the five point harness accommodating wearable blanket in accordance with an embodiment of the present invention. The depiction includes a knee line B in addition to the same reference characters and limitation as described and referenced above in FIG. 1.

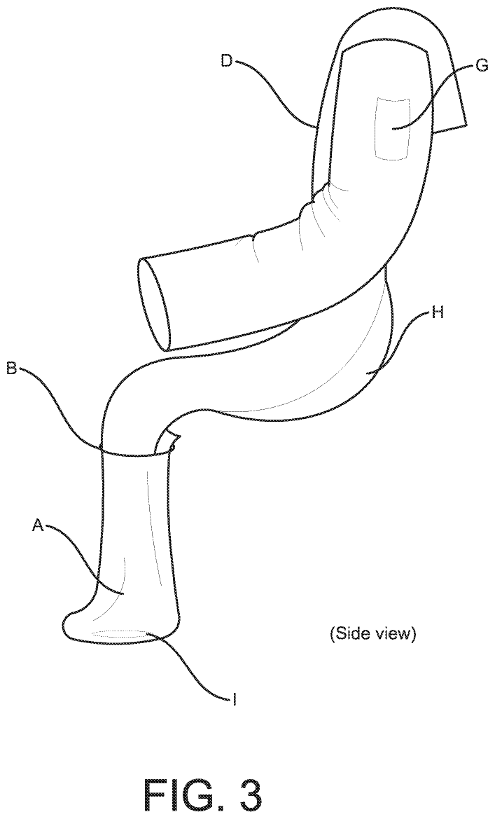

FIG. 3 is a side perspective view of the five point harness accommodating wearable blanket in accordance with an embodiment of the present disclosure. The depiction illustrates how the side flaps H are folded or tucked underneath the bottom and thighs of a wearer and includes the same reference characters and limitation as described and referenced above in FIG. 1.

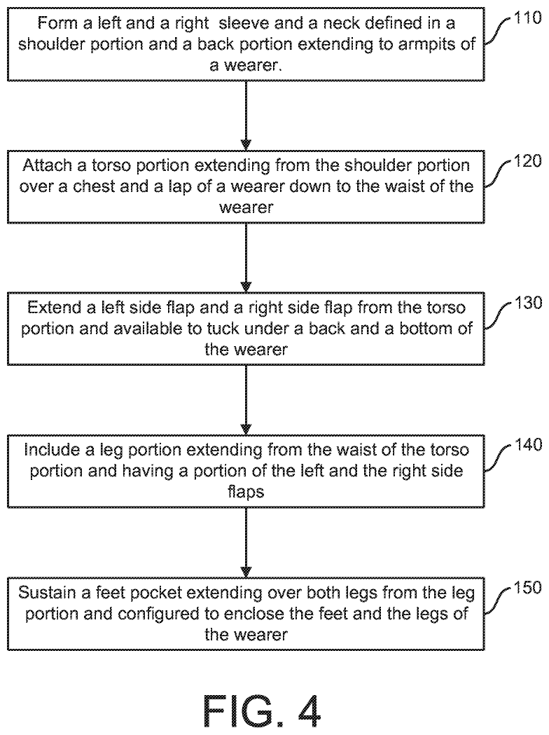

FIG. 4 is a flow chart of a method of wearing the five point harness accommodating wearable blanket in accordance with an embodiment of the present disclosure. The method includes forming 110 a left and a right sleeve and a neck defined in a shoulder portion and a back portion extending to armpits of a wearer. The method also includes attaching 120 a torso portion extending from the shoulder portion over a chest and a lap of a wearer down to the waist of the wearer. The method additionally includes extending 130 a left side flap and a right side flap from the torso portion and available to tuck under a back and a bottom of the wearer. The method further including 140 a leg portion extending from the waist of the torso portion and having a portion of the left and the right side flaps. The method yet includes sustaining 150 a feet pocket extending over both legs from the leg portion and configured to enclose the feet and the legs of the wearer.

Although the operations of the method(s) herein are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

While the forgoing examples are illustrative of the principles of the present disclosure in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the disclosure be limited, except as by the specification and claims set forth herein in a non-provisional specification to follow.

What is claimed is:

1. A blanket comprising:
a wearable blanket accommodating a wearer in a five point harness over both shoulders, around a waist and at the crotch of the wearer, the wearable blanket comprising:
a left and a right sleeve and a neck defined in a shoulder portion and a back portion extending to armpits of a wearer;
a torso portion extending from the shoulder portion over a chest and a lap of a wearer down to the waist of the wearer;
a left side flap and a right side flap extending from the torso portion and available to tuck under a back and a bottom of the wearer;
a leg portion extending from the waist of the torso portion and including a portion of the left and the right side flaps; and
a feet pocket extending over both legs from the leg portion and configured to enclose the feet and the legs of the wearer.

2. The blanket of claim 1, further comprising an elastic attached to the feet pocket, the elastic configured to extend over the knees of the wearer and sustain the feet pocket adjacent the knees.

3. The blanket of claim 1, further comprising a large pocket on the breast of the torso portion.

4. The blanket of claim 1, further comprising a plurality of pockets internal to the feet pocket for an insertion of pads for heels of the wearer.

5. The blanket of claim 1, further comprising a glasses pocket on one of the left and the right sleeves.

6. The blanket of claim 1, further comprising a neck roll accessory for the wearable blanket.

7. The blanket of claim 1, further comprising one of a hook and loop attached adjacent the neck opening for attaching a neck roll accessory via a complementary hook and loop attached to the neck roll.

8. The blanket of claim 1, wherein the blanket comprises one of a warm material for winter wear and a cool material for summer wear.

9. The blanket of claim 1, wherein the left and the right sleeves are long sleeves.

10. The blanket of claim 1, wherein the shoulder portion defines a neck opening with a collar.

11. The blanket of claim 1, wherein the left side flap and the right side flap each resemble half of a heart shape profile.

12. A method of wearing a blanket, the method comprising:
forming a left and a right sleeve and a neck defined in a shoulder portion and a back portion extending to armpits of a wearer;
attaching a torso portion extending from the shoulder portion over a chest and a lap of a wearer down to the waist of the wearer;
extending a left side flap and a right side flap from the torso portion and available to tuck under a back and a bottom of the wearer;
including a leg portion extending from the waist of the torso portion and having a portion of the left and the right side flaps; and
sustaining a feet pocket extending over both legs from the leg portion and configured to enclose the feet and the legs of the wearer.

13. The method of claim 12, further comprising stretching an elastic attached to the feet pocket, the elastic configured to extend over the knees of the wearer and sustain the feet pocket adjacent the knees.

14. The method of claim 12, further comprising attaching a neck roll adjacent the neck opening via a hook and a loop material.

15. The method of claim 12, further comprising tucking the left and the right side flaps under a bottom and under a lower back of the wearer.

16. The method of claim 12, further comprising putting the blanket on a wearer in a five point harness over both shoulders, around a waist and at the crotch of the wearer.

17. The method of claim 12, further comprising inserting a wearer's arms through the sleeves and a wearer's feet into the feet pocket and tucking the flaps under the wearer while in a five point restrained chair.

18. The method of claim 12, further comprising inserting how durometer pads into a plurality of internal pockets in the feet pocket to cushion the wearer's feet.

19. The method of claim 12, further comprising covering a side and a bottom of the wearers thighs via wrapping the side flaps there around.

20. The method of claim 12, further comprising removing a wearer's arms from the sleeves and a wearer's feet from the feet pocket and untucking the flaps from under the wearer while in a five point restrained chair.
